(19) United States
(12) Patent Application Publication
(10) Pub. No.: US 2026/0251593 A1
(43) Pub. Date: Aug. 27, 2026

(54) METHOD AND SYSTEM FOR CORE SAMPLE ANALYSIS, ELECTRONIC DEVICE, AND STORAGE MEDIUM

(71) Applicant: PetroChina Company Limited, Beijing (CN)

(72) Inventors: Guangyou ZHU, Beijing (CN); Zhiyong CHEN, Beijing (CN); Yinghui CAO, Beijing (CN); Debo MA, Beijing (CN); Zhanfeng QIAO, Beijing (CN); Shipeng HUANG, Beijing (CN); Junjia FAN, Beijing (CN); Weiyan CHEN, Beijing (CN); Yunyan NI, Beijing (CN); Jianfeng ZHENG, Beijing (CN); Qiufen LI, Beijing (CN); Chuang LI, Beijing (CN); Ming ZHANG, Beijing (CN)

(21) Appl. No.: 19/137,178

(22) PCT Filed: Dec. 13, 2022
(86) PCT No.: PCT/CN2022/138665

§ 371 Date: Aug. 13, 2025

(30) Foreign Application Priority Data
Dec. 7, 2022 (CN) 202211564009.6

(51) Int. Cl. G01N 23/2206; G01N 23/046; G01N 23/083; G01N 23/223

(52) U.S. Cl. CPC G01N 23/2206; G01N 23/046; G01N 23/083; G01N 23/223; G01N 2223/616

(57) ABSTRACT

Provided in the present invention are a method and system for core sample analysis, an electronic device, and a storage medium. The method comprises steps of: obtaining a dual-energy spiral computed tomography (CT) scan image of a meter-sized large core to be analyzed, wherein the dual-energy spiral CT scan image comprises a low-energy CT scan image and a high-energy CT scan image; determining at least three small core samples in the meter-sized large core to be analyzed; obtaining a micron CT scan image of each of the small core samples and an XRF scan image of a surface feature region; determining the organic matter volume content, porosity, and inorganic mineral volume content of each of the small core samples by using the micron CT scan image and the XRF scan image of the surface feature region of each of the small core samples; and determining the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

### FIELD OF TECHNOLOGY

The present invention relates to the technical field of petroleum well logging, in particular to a method and a system for core sample analysis, an electronic device, and a storage medium.

### BACKGROUND

In the process of oil and gas field exploration, development and evaluation, well logging data is typically used to determine geophysical parameters. The geophysical characteristics such as the electrochemical characteristics, conductivity, acoustic characteristics and radioactivity of rock formations are utilized to identify oil-bearing formations, gas-bearing formations, rock formations, and water-bearing formations, and to specifically determine the properties such as reservoir locations, lithology, and the organic matter abundance and maturity of oil reservoirs. The well logging data can only indirectly and conditionally reflect the geological characteristics of the rock formations. To comprehensively understand the geological features of oil and gas fields and accurately determine and evaluate the oil-bearing formations and the gas-bearing formations, coring operations are typically required. The obtained cores are prepared into samples and the core samples are analyzed with various experimental instruments by using various methods to determine key parameters of source reservoirs.

In a conventional laboratory method for core sample analysis, techniques such as scanning electron microscopy (SEM), backscattered electron imaging (BSE), energy dispersive x-ray spectroscopy (EDS), and combustion methods are commonly used for microscopic mineral component analysis and the organic matter analysis, and methods such as mercury intrusion, seepage, and displacement are commonly used for macroscopic porosity and permeability analysis. The conventional laboratory method for core sample analysis is typically complex, time-consuming, labor-intensive, and inefficient. Moreover, the conventional laboratory method for core sample analysis requires the destruction of the core sample, leading to scattered analysis data and failure to achieve one-to-one correspondence between various property parameters in the same region or on the same sample. Additionally, the conventional laboratory method for core sample analysis involves a large scale gap between microscopic and macroscopic analyses, reducing the significance of results for guiding actual production.

In summary, the efficient and accurate analysis on a core sample without damaging the core sample is one of the issues to be currently addressed.

### SUMMARY

An objective of the present invention is to provide a method and an apparatus capable of efficiently and accurately analyzing a core sample without damaging the core sample to determine key parameters required for oil and gas exploration.

To achieve the above objective, the present invention provides the following technical solutions in four aspects.

In a first aspect, the present invention provides a method for core sample analysis, comprising steps of:
obtaining a dual-energy spiral computed tomography (CT) scan image of a meter-sized large core to be analyzed; determining at least three small core samples in the meter-sized large core to be analyzed, wherein the dual-energy spiral CT scan image comprises a low-energy CT scan image and a high-energy CT scan image;
obtaining an XRF scan image of a surface feature region of each of the small core samples and a micron CT scan image of each of the small core samples;
determining the organic matter volume content (i.e., the volume ratio of the organic matter to the small core sample), porosity (i.e., the volume ratio of pores to the small core sample) and inorganic mineral volume content (i.e., the volume ratio of inorganic mineral to the small core sample) of each of the small core samples by using the micron CT scan image and the XRF scan image of the surface feature region of each of the small core samples; and
determining the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

In a second aspect, the present invention provides a system for core sample analysis, comprising:
a large-core data acquisition module, configured to obtain a dual-energy spiral CT scan image of a meter-sized large core to be analyzed; and to determine at least three small core samples in the meter-sized large core to be analyzed, wherein the dual-energy spiral CT scan image comprises a low-energy CT scan image and a high-energy CT scan image;
a small-core data acquisition module, configured to obtain an XRF scan image of a surface feature region of each of the small core samples and a micron CT scan image of each of the small core samples;
a small-core parameter determination module, configured to determine the organic matter volume content (i.e., the volume ratio of the organic matter to the small core sample), porosity (i.e., the volume ratio of pores to the small core sample) and inorganic mineral volume content (i.e., the volume ratio of inorganic mineral to the small core sample) of each of the small core samples by using the micron CT scan image and the XRF scan image of the surface feature region of each of the small core samples; and
a large-core parameter determination module, configured to determine the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

In a third aspect, the present invention provides an electronic device, comprising a processor, a memory, and a computer program stored in the memory and executable by the processor, wherein the steps of the method for core sample analysis are implemented as the processor executes the program.

In a fourth aspect, the present invention provides a computer-readable storage medium, having a computer program stored therein, wherein the steps of the method for core sample analysis are implemented as the processor executes the program.

The technical solutions provided in the present invention enable the determination of the parameters such as the organic matter volume content distribution, porosity distribution and inorganic mineral volume content distribution in the meter-sized large core without damaging the standard meter-sized large core from the formation on the basis of the core acquisition on site during exploration, significantly improving the accuracy of oil and gas reservoir evaluation and description, and providing a reference basis for exploration and mining. The technical solutions provided in the present invention accelerate the analysis speed of the core after field coring, improve the analysis efficiency, and significantly enhance the analysis accuracy without damaging the core.

### BRIEF DESCRIPTION OF THE DRAWINGS

FIG. 1 is a density curve chart;

FIG. 2 is a relative atomic number curve chart;

FIG. 3 is a schematic diagram demonstrating inorganic mineral analysis in the present invention;

FIG. 4 is a schematic diagram of the organic matter extraction using micron CT (cyan represents organic matter); and

FIG. 5 shows a three-dimensional structural diagram of connected pores in a micron CT core calculated by using the digital core technology and the pore distribution calculation in the embodiments of the present invention.

### DESCRIPTION OF THE EMBODIMENTS

To make the objectives, technical solutions and advantages of the embodiments of the present invention clearer, the technical solutions in the embodiments of the present invention will be described clearly and completely below with reference to the accompanying drawings in the embodiments of the present invention. It is clear that the described embodiments are some but not all of the embodiments of the present invention. All other embodiments obtained by those of ordinary skill in the art based on the embodiments in the present invention without creative efforts shall fall within the protection scope of the present invention.

A specific embodiment of the present invention provides a method for core sample analysis, comprising:
Step S1: obtaining a dual-energy spiral CT scan image of a meter-sized large core to be analyzed; determining at least three small core samples in the meter-sized large core to be analyzed, wherein the dual-energy spiral CT scan image comprises a low-energy CT scan image and a high-energy CT scan image;
Step S2: obtaining an XRF scan image of a surface feature region of each of the small core samples and a micron CT scan image of each of the small core samples;
Step S3: determining the organic matter volume content (i.e., the volume ratio of the organic matter to the small core sample), porosity (i.e., the volume ratio of pores to the small core sample) and inorganic mineral volume content (i.e., the volume ratio of inorganic mineral to the small core sample) of each of the small core samples by using the micron CT scan image and the XRF scan image of the surface feature region of each of the small core samples; and
Step S4: determining the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

In the method for core sample analysis, it is considered that the core samples consist of the organic matter, pores, and inorganic minerals. That is, for a given core sample, the sum of the organic matter volume content, the porosity, and the inorganic mineral volume content is 1.

In an embodiment, the small core samples are centimeter-sized or millimeter-sized.

In an embodiment, the XRF scan image of the small core samples is obtained by scanning with microbeam X-ray fluorescence analysis scanning.

In an embodiment, the micron CT scan image of the small core samples is obtained by dual-energy scanning with a micron X-ray microscope.

In an embodiment, the dual-energy spiral CT scan image of the meter-sized large core to be analyzed is obtained by dual-energy spiral CT scanning with a standard sample.

In an embodiment, the Step S3 of determining the organic matter volume content, porosity, and inorganic mineral volume content of each of the small core samples by using the micron CT scan image and the XRF scan image of the surface feature region of each of the small core samples, comprises:
Step S31: determining the organic matter distribution, porosity distribution and inorganic mineral distribution of each of the small core samples by using the micron CT scan image and the XRF scan image of the surface feature region of each of the small core samples; and
Step S32: determining the organic matter volume content, porosity, and inorganic mineral volume content of each of the small core samples based on the organic matter distribution, porosity distribution and inorganic mineral distribution of each of the small core samples.

Further, in Step S31, the organic matter distribution, porosity distribution and inorganic mineral distribution of each of the small core samples are determined by
Step S311: determining organic matter, pores, and inorganic mineral in the surface feature region of the small core sample based on the micron CT scan image and the XRF scan image of the surface feature region of the small core sample; and
Step S312: extracting the organic matter, the pores, and the inorganic mineral from the micron CT scan image of the small core sample based on the micron CT scan image characteristics of the organic matter, pores and inorganic mineral in the surface feature region of the small core sample, and determining the organic matter distribution, porosity distribution and inorganic mineral distribution of the small core sample.

For example, in Step S312, the organic matter, the pores, and the inorganic mineral can be extracted from the micron CT scan image of the small core sample based on the grayscales of the organic matter, pores and inorganic mineral in the micron CT scan image in the surface feature region of the small core sample, and determining the organic matter distribution, porosity distribution and inorganic mineral distribution of the small core sample.

For example, in Step S311, the organic matter, the pores, and the inorganic mineral in the surface feature region of the small core sample are preliminarily determined based on the micron CT scan image of the surface feature region of the small core sample, then component identification is performed on the preliminarily determined organic matter, pores and inorganic mineral in the surface feature region of the small core sample are identified by using the XRF scan image of the surface feature region of the small core sample, and the organic matter, the pores, and the inorganic mineral in the surface feature region of the small core sample are calibrated, so as to accurately determine the organic matter, the pores, and the inorganic mineral in the surface feature region of the small core sample.

In an embodiment, the Step S4, determining the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples comprises:
Step S41: determining a first CT characterization value and a second CT characterization value of each layer of the meter-sized large core to be analyzed based on the dual-energy spiral CT scan image of the meter-sized large core to be analyzed, wherein the first CT characterization value corresponds to a CT characterization value from the low-energy CT scan image in the dual-energy spiral CT scan image, and the second CT characterization value corresponds to a CT characterization value from the high-energy CT scan image in the dual-energy spiral CT scan image;
Step S42: determining the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value based on the organic matter volume content, porosity, and inorganic mineral volume content of each of the small core samples in combination with the first CT characterization value and the second CT characterization value of each layer of the meter-sized large core to be analyzed corresponding to each of the small core samples; and
Step S43: determining the organic matter volume content and/or porosity and/or inorganic mineral volume content of each layer of the meter-sized large core to be analyzed based on the first CT characterization value and the second CT characterization value of each layer of the meter-sized large core to be analyzed in combination with the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value, and determining the organic matter volume content distribution and/or porosity distribution and/or inorganic mineral volume content distribution in the meter-sized large core to be analyzed.

Further, in Step S42, the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value satisfy the following equations:

$$C {T}_{x} = {X}_{1} \cdot {V}_{1} + {X}_{2} \cdot {V}_{2} + {X}_{3} \cdot {V}_{3}$$

$$C {T}_{Y} = {Y}_{1} \cdot {V}_{1} + {Y}_{2} \cdot {V}_{2} + {Y}_{3} \cdot {V}_{3}$$

where CTₓ is the first CT characterization value; X₁ is the contribution weight of the organic matter content per unit volume to the first CT characterization value; V₁ is the organic matter volume content; X₂ is the contribution weight of the pore content per unit volume to the first CT characterization value; V₂ is the porosity; X₃ is the contribution weight of the inorganic mineral content per unit volume to the first CT characterization value; Vs is the inorganic mineral volume content; CT_{Y} is the second CT characterization value; Y₁ is the contribution weight of the organic matter content per unit volume to the second CT characterization value; Y₂ is the contribution weight of the pore content per unit volume to the second CT characterization value; and Y₃ is the contribution weight of the inorganic mineral content per unit volume to the second CT characterization value.

Further, the first CT characterization value can characterize the relative atomic number, and the second CT characterization value can characterize the average density. For example, the first CT characterization value is a grayscale value, and the second CT characterization value is a grayscale value. For another example, the first CT characterization value is the relative atomic number, and the second CT characterization value is the average density.

In an embodiment, the method further comprises: determining the total organic carbon of each of the small core samples by using the micron CT scan image and the XRF scan image of the surface feature region of each of the small core samples.

Further, the total organic carbon of each of the small core samples is determined by
determining the organic matter in the surface feature region of the small core sample based on the micron CT scan image and the XRF scan image of the surface feature region of the small core sample;
extracting the organic matter from the micron CT scan image of the small core sample based on the micron CT scan image characteristics of the organic matter in the surface feature region of the small core sample, and determining the volume of the organic matter in the small core sample further based on the volume of the small core sample;
determining the density of the organic matter by using the micron CT scan
image of the organic matter in the small core sample;
determining the carbon element mass content of the organic matter based on the XRF scan image of the organic matter in the surface feature region of the small core sample; and
determining the total organic carbon (TOC) of the small core sample by using the volume, density and carbon element mass content of the organic matter in combination with the mass of the small core sample.

Furthermore, the TOC is determined by the following equations:

$$TOC = {m}_{C} \div {m}_{2}$$

$${m}_{O} = {ρ}_{0} \times {V}_{O}$$

$${m}_{C} = {m}_{O} \times {W}_{OC}$$

where TOC is the total organic carbon; m_{C} is the total mass of organic carbon; m_{O} is the total mass of the organic matter; m₂ is the mass of a core sample to be analyzed; ρ_{O} is the density of the organic matter; V_{O} is the volume of the organic matter; and W_{OC} is the carbon element mass content of the organic matter.

The micron CT scan image of the organic matter in the small core sample is used to determine the density of the organic matter by a conventional approach. For example, the density of the organic matter is determined by using the average grayscale of the micron CT scan image of the organic matter in the small core sample in combination with the grayscale of a micron CT scan image of a standard sample.

In an embodiment, the method further comprises: a step of determining the organic matter carbon content distribution in the meter-sized large core to be analyzed by using the micron CT scan image and the XRF scan image of the surface feature region of each of the small core samples in combination with the dual-energy spiral CT scan image of the meter-sized large core to be analyzed and the organic matter volume content distribution in the meter-sized large core to be analyzed.

Further, the step of determining the organic matter carbon content distribution in the meter-sized large core comprises:
determining the organic matter in the surface feature region of each of the small core samples based on the micron CT scan image and the XRF scan image of the surface feature region of each of the small core samples;
determining the density of the organic matter by using the micron CT scan image of the organic matter in each of the small core samples;
determining the carbon element mass content of the organic matter based on the XRF scan image of the organic matter in the surface feature region of each of the small core samples;
determining the average density of each layer of the meter-sized large core to be analyzed by using the dual-energy spiral CT scan image of the meter-sized large core to be analyzed; and
determining the total organic carbon of each layer of the meter-sized large core to be analyzed by using the density and carbon element mass content of the organic matter in combination with the average density and the organic matter volume content of each layer of the meter-sized large core to be analyzed.

Furthermore, the total organic carbon of each layer of the meter-sized large core to be analyzed is determined by the following equation:

$$T O {C}_{i} = {ρ}_{0} \cdot {V}_{1 i} \cdot {W}_{OC} \div {ρ}_{i}$$

where TOCᵢ is the total organic carbon of the i^{th} layer of the meter-sized large core to be analyzed; ρ_{O} is the density of the organic matter; V₁ᵢ is the organic matter volume content of the i^{th} layer of the meter-sized large core to be analyzed; W_{OC} is the carbon element mass content of the organic matter; and ρ₁ is the average density of the i^{th} layer of the meter-sized large core to be analyzed.

The micron CT scan image of the organic matter in the small core sample is used to determine the density of the organic matter by a conventional approach. For example, the density of the organic matter is determined by using the average grayscale of the micron CT scan image of the organic matter in the small core sample in combination with the grayscale of the micron CT scan image of a standard sample.

The dual-energy spiral CT scan image of the meter-sized large core to be analyzed is used to determine the average density of each layer of the meter-sized large core to be analyzed by a conventional approach. For example, the average density of each layer of the meter-sized large core to be analyzed is determined by using the average grayscale of the high-energy CT scan image in the dual-energy spiral CT scan image of each layer of the meter-sized large core to be analyzed in combination with the grayscale of the CT scan image of a standard sample.

In an embodiment, the method further comprises: a step of determining the type of the organic matter by using the micron CT scan image and the XRF scan image of the surface feature region of each of the small core samples.

Further, the type of the organic matter is determined by
determining the organic matter in the surface feature region of each of the small core samples based on the micron CT scan image and the XRF scan image of the surface feature region of each of the small core samples;
determining the carbon element mass content and oxygen element mass content of the organic matter based on the XRF scan image of the organic matter in the surface feature region of each of the small core samples;
determining the number ratio of oxygen atoms to carbon atoms in the organic matter based on the carbon element mass content and oxygen element mass content of the organic matter; and
determining the type of the organic matter by using the number ratio of oxygen atoms to carbon atoms in the organic matter,
wherein the number ratio of oxygen atoms to carbon atoms in the organic matter can be determined by the following equation:

$${R}_{OC} = \frac{{W}_{OO} \cdot 12}{{W}_{OC} \cdot 16}$$

where R_{OC} is the number ratio of oxygen atoms to carbon atoms in the organic matter; W_{OO} is the oxygen element mass content of the organic matter; and W_{OC} is the carbon element mass content of the organic matter.

Typically, the number ratio of oxygen atoms to carbon atoms when the type of the organic matter is Type I kerogen<the number ratio of oxygen atoms to carbon atoms when the type of the organic matter is Type II kerogen<the number ratio of oxygen atoms to carbon atoms when the type of the organic matter is Type III kerogen.

Further, the type of the organic matter is determined by
determining the organic matter in the surface feature region of each of the small core samples based on the micron CT scan image and the XRF scan image of the surface feature region of each of the small core samples;
determining the carbon element mass content and hydrogen element mass content of the organic matter based on the XRF scan image of the organic matter in the surface feature region of each of the small core samples;
determining the number ratio of hydrogen atoms to carbon atoms in the organic matter based on the carbon element mass content and hydrogen element mass content of the organic matter; and
determining the type of the organic matter by using the number ratio of hydrogen atoms to carbon atoms in the organic matter,
wherein the number ratio of hydrogen atoms to carbon atoms in the organic matter can be determined by the following equation:

$${R}_{OH} = \frac{{W}_{OH}}{{W}_{OC} \cdot 16}$$

where R_{OC} is the number ratio of hydrogen atoms to carbon atoms in the organic matter; W_{OH} is the hydrogen element mass content of the organic matter; and W_{OC} is the carbon element mass content of the organic matter.

Typically, the number ratio of hydrogen atoms to carbon atoms when the type of the organic matter is Type I kerogen or Type II kerogen>the number ratio of hydrogen atoms to carbon atoms when the type of the organic matter is Type III kerogen.

In an embodiment, the method further comprises: a step of determining the organic matter maturity by using the micron CT scan image and the XRF scan image of the surface feature region of each of the small core samples.

Further, the organic matter maturity is determined by
determining the organic matter in the surface feature region of each of the small core samples based on the micron CT scan image and the XRF scan image of the surface feature region of each of the small core samples;
determining the elemental composition and the content of each element of the organic matter based on the XRF scan image of the organic matter in the surface feature region of each of the small core samples;
determining the average atomic number of the organic matter based on the elemental composition and the content of each element of the organic matter; and
determining the vitrinite reflectance of the organic matter based on the average atomic number of the organic matter.

Further, the average atomic number of the organic matter is determined by the following equation:

$${Z}_{0} = {[ {{\sum }_{i = 1}}^{N} ( {f}_{i} \cdot {Z}_{i} \cdot {n}_{i} ) ]}^{\frac{1}{A}}$$

where Z₀ is the average atomic number of the organic matter; ƒᵢ is the electron number contribution ratio of an i^{th} constituent element of the organic matter; Zᵢ is the atomic number of the i^{th} constituent element of the organic matter; nᵢ is the number of atoms of the i^{th} constituent element of the organic matter; N is the total number of constituent elements of the organic matter; and A is a coefficient, with a typical value of 3.2.

Further, determining the vitrinite reflectance of the organic matter based on the average atomic number of the organic matter comprises:
obtaining a relational expression between the vitrinite reflectance and the average atomic number of the organic matter, wherein, for example, the vitrinite reflectance of a standard sample and the average atomic number can be used to be fitted to determine the relational expression between the vitrinite reflectance and the average atomic number of the organic matter; and
determining the vitrinite reflectance of the organic matter by using the average atomic number of the organic matter and the relational expression between the vitrinite reflectance and the average atomic number of the organic matter.

Further, the method comprises:
determining whether the core is a source rock based on the organic matter maturity.

In an embodiment, the method further comprises: a step of determining the inorganic mineral composition by using the micron CT scan image and the XRF scan image of the surface feature region of each of the small core samples.

Further, the inorganic mineral composition is determined by
determining various inorganic minerals in the surface feature region of each of the small core samples based on the micron CT scan image and the XRF scan image of the surface feature region of each of the small core samples;
determining the elemental composition and the content of each element of the various inorganic minerals based on XRF scan images of the various inorganic minerals in the surface feature region of each of the small core samples; determining the mineral types of the various inorganic minerals based on the elemental composition and the content of each element of the various inorganic minerals; and
extracting the various inorganic minerals from the micron CT scan image of each of the small core samples based on the micron CT scan image characteristics of the various inorganic minerals in the surface feature region of each of the small core samples, and determining the proportions of the various inorganic minerals in inorganic mineral.

Thus, the determination of the inorganic mineral composition is achieved (the mineral types of the inorganic mineral and the proportions of the various inorganic minerals are determined).

In an embodiment, the method further comprises:
The pore size, pore distribution features and effective porosity (the ratio of the volume of connected pores to the volume of the core) of each of the small core samples are determined based on the micron CT scan image of each of the small core samples.

The embodiments of the present invention further provide a specific implementation of a system for core sample analysis. The system is configured to implement the embodiments of the method for core sample analysis as described above. The system comprises:
a large-core data acquisition module 21, configured to obtain a dual-energy spiral CT scan image of a meter-sized large core to be analyzed; and to determine at least three small core samples in the meter-sized large core to be analyzed, wherein the dual-energy spiral CT scan image comprises a low-energy CT scan image and a high-energy CT scan image;
a small-core data acquisition module 22, configured to obtain an XRF scan image of a surface feature region of each of the small core samples and a micron CT scan image of each of the small core samples;
a small-core parameter determination module 23, configured to determine the organic matter volume content (i.e., the volume ratio of the organic matter to the small core sample), porosity (i.e., the volume ratio of pores to the small core sample) and inorganic mineral volume content (i.e., the volume ratio of inorganic mineral to the small core sample) of each of the small core samples by using the micron CT scan image and the XRF scan image of the surface feature region of each of the small core samples; and
a large-core parameter determination module 24, configured to determine the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

In an embodiment, the small core samples are centimeter-sized or millimeter-sized.

In an embodiment, the XRF scan image of the small core samples is obtained by scanning with microbeam X-ray fluorescence analysis scanning.

In an embodiment, the micron CT scan image of the small core samples is obtained by dual-energy scanning with a micron X-ray microscope.

In an embodiment, the dual-energy spiral CT scan image of the meter-sized large core to be analyzed is obtained by dual-energy spiral CT scanning with a standard sample.

In an embodiment, the small-core parameter determination module 23 comprises:
a first determination submodule 231, configured to determine the organic matter distribution, porosity distribution and inorganic mineral distribution of each of the small core samples by using the micron CT scan image and the XRF scan image of the surface feature region of each of the small core samples; and
a second determination submodule 232, configured to determine the organic matter volume content, porosity, and inorganic mineral volume content of each of the small core samples based on the organic matter distribution, porosity distribution and inorganic mineral distribution of each of the small core samples.

Further, the first determination submodule 231 comprises:
a surface feature region extraction unit 2311, configured to determine organic matter, pores, and inorganic mineral in the surface feature region of the small core sample based on the micron CT scan image and the XRF scan image of the surface feature region of the small core sample; and
a distribution determination unit 2312, configured to extract the organic matter, the pores, and the inorganic mineral from the micron CT scan image of the small core sample based on the micron CT scan image characteristics of the organic matter, pores and inorganic mineral in the surface feature region of the small core sample, and determining the organic matter distribution, porosity distribution and inorganic mineral distribution of the small core sample.

In an embodiment, the large-core parameter determination module 24 comprises:
a CT characterization value determination submodule 241, configured to determine a first CT characterization value and a second CT characterization value of each layer of the meter-sized large core to be analyzed based on the dual-energy spiral CT scan image of the meter-sized large core to be analyzed, wherein the first CT characterization value corresponds to a CT characterization value from the low-energy CT scan image in the dual-energy spiral CT scan image, and the second CT characterization value corresponds to a CT characterization value from the high-energy CT scan image in the dual-energy spiral CT scan image;
a contribution weight determination submodule 242, configured to determine the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value based on the organic matter volume content, porosity, and inorganic mineral volume content of each of the small core samples in combination with the first CT characterization value and the second CT characterization value of each layer of the meter-sized large core to be analyzed corresponding to each of the small core samples; and
a large-core parameter determination submodule 243, configured to determine the organic matter volume content and/or porosity and/or inorganic mineral volume content of each layer of the meter-sized large core to be analyzed based on the first CT characterization value and the second CT characterization value of each layer of the meter-sized large core to be analyzed in combination with the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value, and determining the organic matter volume content distribution and/or porosity distribution and/or inorganic mineral volume content distribution in the meter-sized large core to be analyzed.

Further, the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value satisfy the following equations:

$$C {T}_{x} = {X}_{1} \cdot {V}_{1} + {X}_{2} \cdot {V}_{2} + {X}_{3} \cdot {V}_{3}$$

$$C {T}_{Y} = {Y}_{1} \cdot {V}_{1} + {Y}_{2} \cdot {V}_{2} + {Y}_{3} \cdot {V}_{3}$$

where CTₓ is the first CT characterization value; X₁ is the contribution weight of the organic matter content per unit volume to the first CT characterization value; V₁ is the organic matter volume content; X₂ is the contribution weight of the pore content per unit volume to the first CT characterization value; V₂ is the porosity; X₃ is the contribution weight of the inorganic mineral content per unit volume to the first CT characterization value; V₃ is the inorganic mineral volume content; CT_{Y} is the second CT characterization value; Y₁ is the contribution weight of the organic matter content per unit volume to the second CT characterization value; Y₂ is the contribution weight of the pore content per unit volume to the second CT characterization value; and Y₃ is the contribution weight of the inorganic mineral content per unit volume to the second CT characterization value.

Further, the first CT characterization value can characterize the relative atomic number, and the second CT characterization value can characterize the average density. For example, the first CT characterization value is a grayscale value, and the second CT characterization value is a grayscale value. For another example, the first CT characterization value is the relative atomic number, and the second CT characterization value is the average density.

In an embodiment, the system further comprises:
a small-core TOC determination module 25, configured to determine the total organic carbon of each of the small core samples by using the micron CT scan image and the XRF scan image of the surface feature region of each of the small core samples.

Further, the small-core TOC determination module 25 is specifically configured to determine the total organic carbon of each of the small core samples by:
determining the organic matter in the surface feature region of a small core sample based on the micron CT scan image and the XRF scan image of the surface feature region of the small core sample;
extracting the organic matter from the micron CT scan image of the small core sample based on the micron CT scan image characteristics of the organic matter in the surface feature region of the small core sample, and determining the volume of the organic matter in the small core sample further based on the volume of the small core sample;
determining the density of the organic matter by using the micron CT scan image of the organic matter in the small core sample;
determining the carbon element mass content of the organic matter based on the XRF scan image of the organic matter in the surface feature region of the small core sample; and
determining the total organic carbon (TOC) of the small core sample by using the volume, density and carbon element mass content of the organic matter in combination with the mass of the small core sample.

Furthermore, the TOC is determined by the following equations:

$$TOC = {m}_{C} \div {m}_{2}$$

$${m}_{O} = {ρ}_{0} \times {V}_{O}$$

$${m}_{C} = {m}_{O} \times {W}_{OC}$$

where TOC is the total organic carbon; m_{C} is the total mass of organic carbon; m_{O} is the total mass of the organic matter; m₂ is the mass of a core sample to be analyzed; ρ₀ is the density of the organic matter; V_{O} is the volume of the organic matter; and W_{OC} is the carbon element mass content of the organic matter.

In an embodiment, the system further comprises:
a TOC distribution determination module 26, configured to determine organic matter carbon content distribution in the meter-sized large core to be analyzed by using the micron CT scan image and the XRF scan image of the surface feature region of each of the small core samples in combination with the dual-energy spiral CT scan image of the meter-sized large core to be analyzed and the organic matter volume content distribution in the meter-sized large core to be analyzed.

Further, the TOC distribution determination module 26 is configured to determine the organic matter carbon content distribution in the meter-sized large core to be analyzed by:
determining the organic matter in the surface feature region of each of the small core samples based on the micron CT scan image and the XRF scan image of the surface feature region of each of the small core samples;
determining the density of the organic matter by using the micron CT scan image of the organic matter in each of the small core samples;
determining the carbon element mass content of the organic matter based on the XRF scan image of the organic matter in the surface feature region of each of the small core samples;
determining the average density of each layer of the meter-sized large core to be analyzed by using the dual-energy spiral CT scan image of the meter-sized large core to be analyzed; and
determining the total organic carbon of each layer of the meter-sized large core to be analyzed by using the density and carbon element mass content of the organic matter in combination with the average density and the organic matter volume content of each layer of the meter-sized large core to be analyzed.

Furthermore, the total organic carbon of each layer of the meter-sized large core to be analyzed is determined by the following equation:

$$TO {C}_{i} = {ρ}_{0} \cdot {V}_{1 i} \cdot {W}_{OC} \div {ρ}_{i}$$

where TOCᵢ is the total organic carbon of the i^{th} layer of the meter-sized large core to be analyzed; ρ_{O} is the density of the organic matter; V₁ᵢ is the organic matter volume content of the i^{th} layer of the meter-sized large core to be analyzed; W_{OC} is the carbon element mass content of the organic matter; and ρᵢ is the average density of the i^{th} layer of the meter-sized large core to be analyzed.

In an embodiment, the system further comprises:
a determination module of the type of the organic matter 27, configured to determine the type of the organic matter by using the micron CT scan image and the XRF scan image of the surface feature region of each of the small core samples.

Further, the determination module of the type of the organic matter 27 is configured to determine the type of the organic matter as follows:
determining the organic matter in the surface feature region of each of the small core samples based on the micron CT scan image and the XRF scan image of the surface feature region of each of the small core samples;
determining the carbon element mass content and oxygen element mass content of the organic matter based on the XRF scan image of the organic matter in the surface feature region of each of the small core samples;
determining the number ratio of oxygen atoms to carbon atoms in the organic matter based on the carbon element mass content and oxygen element mass content of the organic matter; and
determining the type of the organic matter by using the number ratio of oxygen atoms to carbon atoms in the organic matter,
wherein the number ratio of oxygen atoms to carbon atoms in the organic matter can be determined by the following equation:

$${R}_{OH} = \frac{{W}_{OH} \cdot 12}{{W}_{OC} \cdot 16}$$

where R_{OC} is the number ratio of oxygen atoms to carbon atoms in the organic matter; W_{OO} is the oxygen element mass content of the organic matter; and W_{OC} is the carbon element mass content of the organic matter.

Typically, the number ratio of oxygen atoms to carbon atoms when the type of the organic matter is Type I kerogen<the number ratio of oxygen atoms to carbon atoms when the type of the organic matter is Type II kerogen<the number ratio of oxygen atoms to carbon atoms when the type of the organic matter is Type III kerogen.

Further, the determination module of the type of the organic matter 27 is configured to determine the type of the organic matter as follows:
determining the organic matter in the surface feature region of each of the small core samples based on the micron CT scan image and the XRF scan image of the surface feature region of each of the small core samples;
determining the carbon element mass content and hydrogen element mass content of the organic matter based on the XRF scan image of the organic matter in the surface feature region of each of the small core samples;
determining the number ratio of hydrogen atoms to carbon atoms in the organic matter based on the carbon element mass content and hydrogen element mass content of the organic matter; and
determining the type of the organic matter by using the number ratio of hydrogen atoms to carbon atoms in the organic matter,
wherein the number ratio of hydrogen atoms to carbon atoms in the organic matter can be determined by the following equation:

$${R}_{OH} = \frac{{W}_{OH}}{{W}_{OC} \cdot 16}$$

where R_{OC} is the number ratio of hydrogen atoms to carbon atoms in the organic matter; W_{OH} is the hydrogen element mass content of the organic matter; and W_{OC} is the carbon element mass content of the organic matter.

Typically, the number ratio of hydrogen atoms to carbon atoms when the type of the organic matter is Type I kerogen or Type II kerogen>the number ratio of hydrogen atoms to carbon atoms when the type of the organic matter is Type III kerogen.

In an embodiment, the system further comprises:
an organic matter maturity determination module 28, configured to determine the organic matter maturity by using the micron CT scan image and the XRF scan image of the surface feature region of each of the small core samples.

Further, the organic matter maturity determination module 28 is configured to determine the organic matter maturity as follows:
determining the organic matter in the surface feature region of each of the small core samples based on the micron CT scan image and the XRF scan image of the surface feature region of each of the small core samples;
determining the elemental composition and the content of each element of the organic matter based on the XRF scan image of the organic matter in the surface feature region of each of the small core samples;
determining the average atomic number of the organic matter based on the elemental composition and the content of each element of the organic matter; and
determining the vitrinite reflectance of the organic matter based on the average atomic number of the organic matter.

Further, the average atomic number of the organic matter is determined by the following equation:

$${Z}_{0} = {[ {\sum }_{i = 1}^{N} ( {f}_{i} \cdot {Z}_{i} \cdot {n}_{i} ) ]}^{\frac{1}{A}}$$

where Z₀ is the average atomic number of the organic matter; ƒᵢ is the electron number contribution ratio of an i^{th} constituent element of the organic matter; is the atomic number of the i^{th} constituent element of the organic matter; nᵢ is the number of atoms of the i^{th} constituent element of the organic matter; N is the total number of constituent elements of the organic matter; and A is a coefficient, with a typical value of 3.2.

Further, the organic matter maturity determination module 28 is configured to determine the average atomic number of the organic matter as follows:
obtaining a relational expression between the vitrinite reflectance and the average atomic number of the organic matter, wherein, for example, the vitrinite reflectance of a standard sample and the average atomic number can be used to be fitted to determine the relational expression between the vitrinite reflectance and the average atomic number of the organic matter; and
determining the vitrinite reflectance of the organic matter by using the average atomic number of the organic matter and the relational expression between the vitrinite reflectance and the average atomic number of the organic matter.

Further, the system further comprises:
a source rock determination module 29, configured to determine whether the core is a source rock based on the organic matter maturity.

In an embodiment, the system further comprises:
an inorganic mineral composition determination module 30, configured to determine the inorganic mineral composition by using the micron CT scan image and the XRF scan image of the surface feature region of each of the small core samples.

Further, the inorganic mineral composition determination module 30 is configured to determine the inorganic mineral composition as follows:
determining various inorganic minerals in the surface feature region of each of the small core samples based on the micron CT scan image and the XRF scan image of the surface feature region of each of the small core samples;
determining the elemental composition and the content of each element of the various inorganic minerals based on XRF scan images of the various inorganic minerals in the surface feature region of each of the small core samples; determining the mineral types of the various inorganic minerals based on the elemental composition and the content of each element of the various inorganic minerals; and
extracting the various inorganic minerals from the micron CT scan image of each of the small core samples based on the micron CT scan image characteristics of the various inorganic minerals in the surface feature region of each of the small core samples, and determining the proportions of the various inorganic minerals in inorganic mineral.

Thus, the determination of the inorganic mineral composition is achieved (the mineral types of the inorganic mineral and the proportions of the various inorganic minerals are determined).

In an embodiment, the system further comprises:
a pore parameter determination module 31, configured to determine the pore size, pore distribution features and effective porosity (the ratio of the volume of connected pores to the volume of the core) of each of the small core samples based on the micron CT scan image of each of the small core samples.

The embodiments of the present invention further provide a specific implementation of an electronic device capable of implementing all the steps in the method for core sample analysis in the above embodiments. The electronic device specifically comprises the following components:
a processor, a memory, a communication interface, and a bus.

The processor, the memory, and the communication interface communicate with each other via the bus. The communication interface is configured to achieve information transmission between related devices such as a server-side device and a client-side device. The processor is configured to invoke a computer program in the memory. When the processor executes the computer program, all the steps in the method for core sample analysis in the above embodiments are implemented. For example, when the processor executes the computer program, the following steps are implemented:
Step S1: obtaining a dual-energy spiral CT scan image of a meter-sized large core to be analyzed; determining at least three small core samples in the meter-sized large core to be analyzed, wherein the dual-energy spiral CT scan image comprises a low-energy CT scan image and a high-energy CT scan image;
Stop S2: obtaining an XRF scan image of a surface feature region of each of the small core samples and a micron CT scan image of each of the small core samples;
Step S3: determining the organic matter volume content (i.e., the volume ratio of the organic matter to the small core sample), porosity (i.e., the volume ratio of pores to the small core sample) and inorganic mineral volume content (i.e., the volume ratio of inorganic mineral to the small core sample) of each of the small core samples by using the micron CT scan image and the XRF scan image of the surface feature region of each of the small core samples; and
Step S4: determining the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

The embodiments of the present invention further provide a computer-readable storage medium capable of implementing all the steps in the method for core sample analysis in the above embodiments. The computer-readable storage medium stores a computer program. When the computer program is executed by a processor, all the steps of the method for core sample analysis in the above embodiments are implemented. For example, when the processor executes the computer program, the following steps are implemented:
Step S1: obtaining a dual-energy spiral CT scan image of a meter-sized large core to be analyzed; determining at least three small core samples in the meter-sized large core to be analyzed, wherein the dual-energy spiral CT scan image comprises a low-energy CT scan image and a high-energy CT scan image;
Step S2: obtaining an XRF scan image of a surface feature region of each of the small core samples and a micron CT scan image of each of the small core samples;
Step S3: determining the organic matter volume content (i.e., the volume ratio of the organic matter to the small core sample), porosity (i.e., the volume ratio of pores to the small core sample) and inorganic mineral volume content (i.e., the volume ratio of inorganic mineral to the small core sample) of each of the small core samples by using the micron CT scan image and the XRF scan image of the surface feature region of each of the small core samples; and
Step S4: determining the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

### Example 1

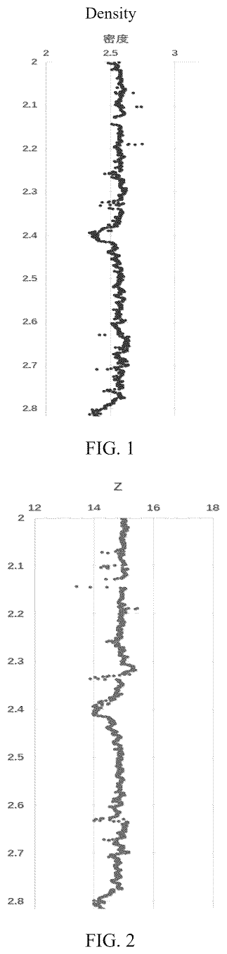

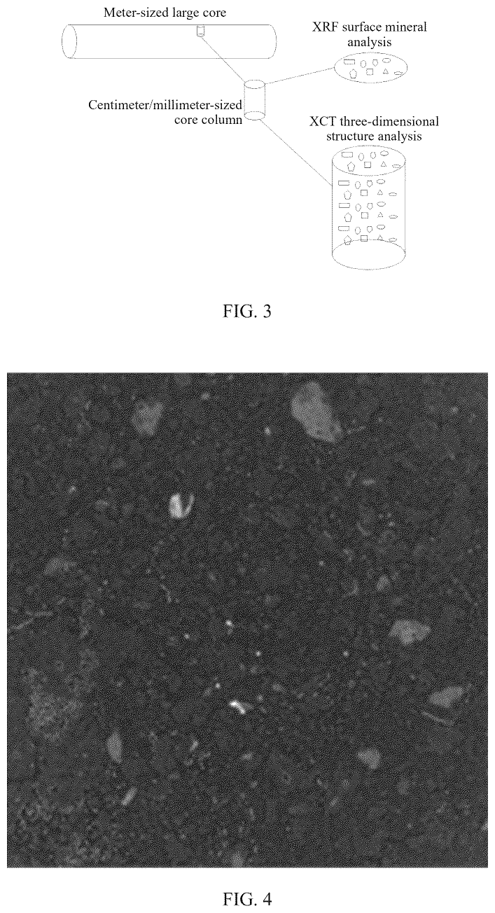

This example provides a method for core sample analysis. The method is configured to analyze the meter-sized core of a source rock, and specifically comprises:
A: performing dual-energy spiral CT scanning with a standard sample on a meter-sized large core to be analyzed of a source rock to obtain a dual-energy spiral CT scan image of each layer of the meter-sized large core to be analyzed; and determining the relative atomic number and average density of each layer of the meter-sized large core to be analyzed based on the dual-energy spiral CT scan image of the meter-sized large core to be analyzed, wherein the relative atomic number corresponds to a CT characterization value of a low-energy CT scan image in the dual-energy spiral CT scan image, and the average density corresponds to the CT characterization value of a high-energy CT scan image in the dual-energy spiral CT scan image (as shown in FIG. 1 and FIG. 2);
wherein, due to the influence of lithology, organic matter content, pore features, and crack features, the average densities and relative atomic numbers of different layers of the meter-sized large core to be analyzed exhibit significant variations; and determining a typical rock section in the meter-sized large core to be analyzed based on the average density and relative atomic number of each layer of the meter-sized large core to be analyzed, and determining a plurality of small core samples (centimeter-sized or millimeter-sized) from the typical rock section;
B: obtaining the mass, volume and density of each of the small core samples;
C: performing feature region scanning on the surface of each of the small core samples through Micron XRF analysis to obtain an XRF scan image of the surface feature region of each of the small core samples; and performing dual-energy scanning on each of the small core samples by a micron X-ray microscope (Micron CT) to obtain a micron CT scan image of each of the small core samples;
D: determining the elemental composition of the surface feature region of each of the small core samples and the mass fraction of each element (including primary organic matter elements such as carbon W_{C}, sulfur W_{S}, nitrogen W_{N}, and oxygen W_{O}, and trace elements such as iron W_{Fe}, copper W_{Cu}, magnesium W_{Mg}, zinc W_{Zn}, molybdenum W_{Mu}, nickel W_{Ni}, and mercury W_{Hg}) based on the XRF scan image of the surface feature region of each of the small core samples;
E: determining the organic matter (as shown in FIG. 4), pores, and inorganic mineral in the surface feature region of each of the small core samples based on the micron CT scan image and the XRF scan image of the surface feature region of each of the small core samples; extracting the organic matter, the pores, and the inorganic mineral from the micron CT scan image of each of the small core samples based on the micron CT scan image characteristics of the organic matter, pores and inorganic mineral in the surface feature region of each of the small core samples to determine the organic matter distribution, porosity distribution and inorganic mineral distribution of each of the small core samples;
determining the volume of the organic matter, the volume of the pores, and the volume of the inorganic mineral in each of the small core samples based on results of the extraction of the organic matter, the pores, and the inorganic mineral from the micron CT scan image of each of the small core samples in combination with the volume of each of the small core samples, and determining the organic matter volume content, porosity, and inorganic mineral volume content of each of the small core samples;
determining the organic matter density (based on a scanned standard sample), mass and relative atomic number (based on a scanned standard sample) of each of the small core samples by using the average grayscale of the micron CT scan image of the organic matter of each of the small core samples, and determining the average density and average relative atomic number of the organic matter through weighted averaging to serve as the organic matter density and the organic matter relative atomic number of the meter-sized large core to be analyzed; and
determining the mass contents of carbon, oxygen, sulfur, and nitrogen in the organic matter of each of the small core samples based on the XRF scan image of the organic matter in the surface feature region of each of the small core samples, and determining the average mass content of carbon, average mass content of oxygen, average mass content of sulfur and average mass content of nitrogen in the organic matter through weighted averaging to serve as the mass contents of carbon, oxygen, sulfur, and nitrogen in the organic matter of the meter-sized large core to be analyzed;
F: determining a first CT grayscale value and a second CT grayscale value of each layer of the meter-sized large core to be analyzed based on the dual-energy spiral CT scan image of the meter-sized large core to be analyzed, wherein the first CT grayscale value corresponds to a CT grayscale value from the low-energy CT scan image in the dual-energy spiral CT scan image, and the second CT grayscale value corresponds to a CT grayscale value from the high-energy CT scan image in the dual-energy spiral CT scan image;
determining the contribution weight of the organic matter content per unit volume to the first CT grayscale value, the contribution weight of the pore content per unit volume to the first CT grayscale value, the contribution weight of the inorganic mineral content per unit volume to the first CT grayscale value, the contribution weight of the organic matter content per unit volume to the second CT grayscale value, the contribution weight of the pore content per unit volume to the second CT grayscale value, and the contribution weight of the inorganic mineral content per unit volume to the second CT grayscale value based on the organic matter volume content, porosity, and inorganic mineral volume content of each of the small core samples in combination with the first CT grayscale value and the second CT grayscale value of each layer of the meter-sized large core to be analyzed corresponding to each of the small core samples, wherein the contribution weight of the organic matter content per unit volume to the first CT grayscale value, the contribution weight of the pore content per unit volume to the first CT grayscale value, the contribution weight of the inorganic mineral content per unit volume to the first CT grayscale value, the contribution weight of the organic matter content per unit volume to the second CT grayscale value, the contribution weight of the pore content per unit volume to the second CT grayscale value, and the contribution weight of the inorganic mineral content per unit volume to the second CT grayscale value satisfy the following equations:

$${CT}_{x} = {X}_{1} \cdot {V}_{1} + {X}_{2} \cdot {V}_{2} - {X}_{3} \cdot {V}_{3}$$

$$C {T}_{Y} = {Y}_{1} \cdot {V}_{1} + {Y}_{2} \cdot {V}_{2} - {Y}_{3} \cdot {V}_{3}$$

where CTₓ is the first CT grayscale value; X₁ is the contribution weight of the organic matter content per unit volume to the first CT grayscale value; V₁ is the organic matter volume content; X₂ is the contribution weight of the pore content per unit volume to the first CT grayscale value; V₂ is the porosity; X₃ is the contribution weight of the inorganic mineral content per unit volume to the first CT grayscale value; V₃ is the inorganic mineral volume content; CT_{Y} is the second CT grayscale value; Y₁ is the contribution weight of the organic matter content per unit volume to the second CT grayscale value; Y₂ is the contribution weight of the pore content per unit volume to the second CT grayscale value; and Y₃ is the contribution weight of the inorganic mineral content per unit volume to the second CT grayscale value; and
determining the organic matter volume content, porosity, and inorganic mineral volume content of each layer of the meter-sized large core to be analyzed based on the first CT grayscale value and the second CT grayscale value of each layer of the meter-sized large core to be analyzed in combination with the contribution weight of the organic matter content per unit volume to the first CT grayscale value, the contribution weight of the pore content per unit volume to the first CT grayscale value, the contribution weight of the inorganic mineral content per unit volume to the first CT grayscale value, the contribution weight of the organic matter content per unit volume to the second CT grayscale value, the contribution weight of the pore content per unit volume to the second CT grayscale value, and the contribution weight of the inorganic mineral content per unit volume to the second CT grayscale value, and determining the organic matter volume content distribution, porosity distribution and inorganic mineral volume content distribution in the meter-sized large core to be analyzed;
G: for each of the small core samples, determining the total organic carbon (TOC) of each of the small core samples by using the volume, density and carbon element mass content of the organic matter in combination with the mass of the core sample according to the following equations:

$$TOC = {m}_{C} \div {m}_{2}$$

$${m}_{O} = {p}_{0} \times {V}_{O}$$

$${m}_{C} = {m}_{O} \times {V}_{OC}$$

where TOC is the total organic carbon; m_{C} is the total mass of organic carbon; m_{O} is the total mass of the organic matter; m₂ is the mass of a core sample to be analyzed; ρ_{O} is the density of the organic matter; V_{O} is the volume of the organic matter; and W_{OC} is the carbon element mass content of the organic matter;
H: determining the total organic carbon of each layer of the meter-sized large core to be analyzed by using the density and carbon element mass content of the organic matter in the meter-sized large core to be analyzed in combination with the average density and the organic matter volume content of each layer of the meter-sized large core to be analyzed according to the following equation:

$${TOC}_{i} = {ρ}_{0} \cdot {V}_{1 i} \cdot {W}_{OC} \div {ρ}_{i}$$

where TOCᵢ is the total organic carbon of the i^{th} layer of the meter-sized large core to be analyzed; ρ_{O} is the density of the organic matter; V₁ᵢ is the organic matter volume content of the i^{th} layer of the meter-sized large core to be analyzed; W_{OC} is the carbon element mass content of the organic matter; and p; is the average density of the i^{th} layer of the meter-sized large core to be analyzed;
I: determining the number ratio of oxygen atoms to carbon atoms in the organic matter based on the carbon element mass content and oxygen element mass content of the organic matter in the meter-sized large core to be analyzed; and determining the type of the organic matter by using the number ratio of oxygen atoms to carbon atoms in the organic matter, wherein the number ratio of oxygen atoms to carbon atoms in the organic matter can be determined by the following equation:

$${R}_{OC} = \frac{{W}_{OO} \cdot 12}{{W}_{OC} \cdot 16} ,$$

where R_{OC} is the number ratio of oxygen atoms to carbon atoms in the organic matter; W_{OO} is the oxygen element mass content of the organic matter; and W_{OC} is the carbon element mass content of the organic matter;
J: determining the average atomic number of the organic matter based on the elemental composition of the organic matter in the meter-sized large core to be analyzed and the content of each element; obtaining a relational expression between the vitrinite reflectance and the average atomic number of the organic matter; determining the vitrinite reflectance of the organic matter by using the average atomic number of the organic matter and the relational expression between the vitrinite reflectance and the average atomic number of the organic matter, wherein the average atomic number of the organic matter is determined by the following equation:

$${Z}_{0} = {[ {\sum }_{i = 1}^{N} ( {f}_{i} \cdot {Z}_{i} \cdot {n}_{i} ) ]}^{\frac{1}{A}} ,$$

where Z₀ is the average atomic number of the organic matter; ƒᵢ is the electron number contribution ratio of an i^{th} constituent element of the organic matter; Zᵢ is the atomic number of the i^{th} constituent element of the organic matter; nᵢ is the number of atoms of the i^{th} constituent element of the organic matter; N is the total number of constituent elements of the organic matter; and A is a coefficient, with a value of 3.2; and
determining whether the core is a source rock based on the organic matter maturity;
K: determining various inorganic minerals in the surface feature region of each of the small core samples based on the micron CT scan image and the XRF scan image of the surface feature region of each of the small core samples; determining the elemental composition and the content of each element of the various inorganic minerals (including trace elements such as iron, copper, magnesium, zinc, molybdenum, nickel, and mercury) based on XRF scan images of the various inorganic minerals in the surface feature region of each of the small core samples; determining the mineral types of the various inorganic minerals based on the elemental composition and the content of each element of the various inorganic minerals; and extracting the various inorganic minerals from the micron CT scan image of each of the small core samples based on the micron CT scan image characteristics of the various inorganic minerals in the surface feature region of each of the small core samples, and determining the proportions of the various inorganic minerals in inorganic mineral, so that the determination of the inorganic mineral composition is achieved (the mineral types of the inorganic mineral and the proportions of the various inorganic minerals are determined); and
L: for each of the small core samples, determining various inorganic minerals in the surface feature region based on the micron CT scan image and the XRF scan image of the surface feature region, and extracting the various minerals from the micron CT scan image based on the micron CT scan image characteristics of the various minerals in the surface feature region to determine inorganic mineral distribution (as shown in FIG. 3).

### Example 2

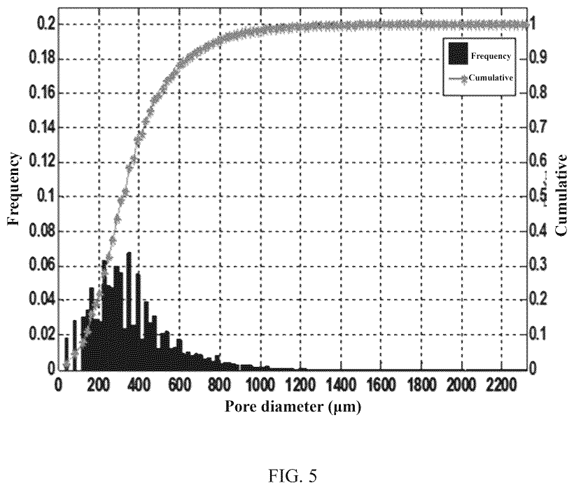

This example provides a method for core sample analysis. The method is configured to analyze the meter-sized core of a reservoir, and specifically comprises:
A: performing dual-energy spiral CT scanning with a standard sample on a meter-sized large core to be analyzed of a reservoir to obtain a dual-energy spiral CT scan image of each layer of the meter-sized large core to be analyzed; and determining the relative atomic number and average density of each layer of the meter-sized large core to be analyzed based on the dual-energy spiral CT scan image of the meter-sized large core to be analyzed, wherein the relative atomic number corresponds to the CT characterization value of a low-energy CT scan image in the dual-energy spiral CT scan image, and the average density corresponds to the CT characterization value of a high-energy CT scan image in the dual-energy spiral CT scan image; and
determining a typical rock section in the meter-sized large core to be analyzed based on the average density and relative atomic number of each layer of the meter-sized large core to be analyzed, and determining a plurality of small core samples (centimeter-sized or millimeter-sized) from the typical rock section;
B: obtaining the mass, volume and density of each of the small core samples;
C: performing feature region scanning on the surface of each of the small core samples through Micron XRF analysis to obtain an XRF scan image of the surface feature region of each of the small core samples; and performing dual-energy scanning on each of the small core samples by a micron X-ray microscope (Micron CT) to obtain a micron CT scan image of each of the small core samples;
D: determining the elemental composition of the surface feature region of each of the small core samples and the mass fraction of each element (including primary organic matter elements such as carbon W_{C}, sulfur W_{S}, nitrogen W_{N}, and oxygen W_{O}, and trace elements such as iron W_{Fe}, copper W_{Cu}, magnesium W_{Mg}, zinc W_{Zn}, molybdenum W_{Mu}, nickel W_{Ni}, and mercury W_{Hg}) based on the XRF scan image of the surface feature region of each of the small core samples;
E: determining organic matter, pores, and inorganic mineral in the surface feature region of each of the small core samples based on the micron CT scan image and the XRF scan image of the surface feature region of each of the small core samples; extracting the organic matter, the pores, and the inorganic mineral from the micron CT scan image of each of the small core samples based on the micron CT scan image characteristics of the organic matter, pores and inorganic mineral in the surface feature region of each of the small core samples to determine the organic matter distribution, porosity distribution and inorganic mineral distribution of each of the small core samples;
determining the volume of the organic matter, the volume of the pores, and the volume of the inorganic mineral in each of the small core samples based on results of the extraction of the organic matter, the pores, and the inorganic mineral from the micron CT scan image of each of the small core samples in combination with the volume of each of the small core samples, and determining the organic matter volume content, porosity, and inorganic mineral volume content of each of the small core samples;
determining the organic matter density (based on a scanned standard sample), mass and relative atomic number (based on a scanned standard sample) of each of the small core samples by using the average grayscale of a micron CT scan image of the organic matter of each of the small core samples, and determining the average density and average relative atomic number of the organic matter through weighted averaging to serve as the organic matter density and the organic matter relative atomic number of the meter-sized large core to be analyzed; and
determining the mass contents of carbon, oxygen, sulfur, and nitrogen in the organic matter of each of the small core samples based on the XRF scan image of the organic matter in the surface feature region of each of the small core samples, and determining the average mass content of carbon, average mass content of oxygen, average mass content of sulfur and average mass content of nitrogen in the organic matter through weighted averaging to serve as the mass contents of carbon, oxygen, sulfur, and nitrogen in the organic matter of the meter-sized large core to be analyzed;
F: determining the contribution weight of the organic matter content per unit volume to the relative atomic number, the contribution weight of the pore content per unit volume to the relative atomic number, the contribution weight of the inorganic mineral content per unit volume to the relative atomic number, the contribution weight of the organic matter content per unit volume to the average density, the contribution weight of the pore content per unit volume to the average density, and the contribution weight of the inorganic mineral content per unit volume to the average density based on the organic matter volume content, porosity, and inorganic mineral volume content of each of the small core samples in combination with the relative atomic number and the average density of each layer of the meter-sized large core to be analyzed corresponding to each of the small core samples, wherein the contribution weight of the organic matter content per unit volume to the relative atomic number, the contribution weight of the pore content per unit volume to the relative atomic number, the contribution weight of the inorganic mineral content per unit volume to the relative atomic number, the contribution weight of the organic matter content per unit volume to the average density, the contribution weight of the pore content per unit volume to the average density, and the contribution weight of the inorganic mineral content per unit volume to the average density satisfy the following equations:

$$C {T}_{x} = {X}_{1} \cdot {V}_{1} + {X}_{2} \cdot {V}_{2} + {X}_{3} \cdot {V}_{3}$$

$${CT}_{Y} = {Y}_{1} \cdot {V}_{1} + {Y}_{2} \cdot {V}_{2} + {Y}_{3} \cdot {V}_{3}$$

where CTₓ is the relative atomic number; X₁ is the contribution weight of the organic matter content per unit volume to the relative atomic number; V₁ is the organic matter volume content; X₂ is the contribution weight of the pore content per unit volume to the relative atomic number; V₂ is the porosity; X₃ is the contribution weight of the inorganic mineral content per unit volume to the relative atomic number; V₃ is the inorganic mineral volume content; CT_{Y} is the average density; Y₁ is the contribution weight of the organic matter content per unit volume to the average density; Y₂ is the contribution weight of the pore content per unit volume to the average density; and Y₃ is the contribution weight of the inorganic mineral content per unit volume to the average density; and
determining the organic matter volume content and/or porosity and/or inorganic mineral volume content of each layer of the meter-sized large core to be analyzed based on the relative atomic number and the average density of each layer of the meter-sized large core to be analyzed in combination with the contribution weight of the organic matter content per unit volume to the relative atomic number, the contribution weight of the pore content per unit volume to the relative atomic number, the contribution weight of the inorganic mineral content per unit volume to the relative atomic number, the contribution weight of the organic matter content per unit volume to the average density, the contribution weight of the pore content per unit volume to the average density, and the contribution weight of the inorganic mineral content per unit volume to the average density, and determining the organic matter volume content distribution and/or porosity distribution and/or inorganic mineral volume content distribution in the meter-sized large core to be analyzed;
G: determining the pore size, pore distribution features (distribution of a given core sample is as shown in FIG. 5) and effective porosity (the ratio of the volume of connected pores to the volume of the core) of each of the small core samples based on the micron CT scan image of each of the small core samples;
F: determining the average atomic number of the organic matter based on the elemental composition of the organic matter in the meter-sized large core to be analyzed and the content of each element; obtaining a relational expression between the vitrinite reflectance and the average atomic number of the organic matter; determining the vitrinite reflectance of the organic matter by using the average atomic number of the organic matter and the relational expression between the vitrinite reflectance and the average atomic number of the organic matter, wherein the average atomic number of the organic matter is determined by the following equation:

$${Z}_{0} = {[ {\sum }_{i = 1}^{N} ( {f}_{i} \cdot {Z}_{i} \cdot {n}_{i} ) ]}^{\frac{1}{A}} ,$$

where Z₀ is the average atomic number of the organic matter; ƒᵢ is the electron number contribution ratio of an i^{th} constituent element of the organic matter; Zᵢ is the atomic number of the i^{th} constituent element of the organic matter; nᵢ is the number of atoms of the i^{th} constituent element of the organic matter; N is the total number of constituent elements of the organic matter; and A is a coefficient, with a value of 3.2;
G: for each of the core samples to be analyzed, determining the lithofacies in the surface feature region based on the micron CT scan image and the XRF scan image of the surface feature region; determining the elemental composition of the lithofacies and the content of each element (including trace elements such as iron, copper, magnesium, zinc, molybdenum, nickel, and mercury) based on the XRF scan image of the lithofacies in the surface feature region; determining the mineral composition based on the elemental composition of the lithofacies and the content of each element; and extracting various minerals from the CT scan image based on CT scan image features of the various minerals in the surface feature region to determine mineral distribution;
H: determining various inorganic minerals in the surface feature region of each of the small core samples based on the micron CT scan image and the XRF scan image of the surface feature region of each of the small core samples; determining the elemental composition and the content of each element of the various inorganic minerals (including trace elements such as iron, copper, magnesium, zinc, molybdenum, nickel, and mercury) based on XRF scan images of the various inorganic minerals in the surface feature region of each of the small core samples; determining the mineral types of the various inorganic minerals based on the elemental composition and the content of each element of the various inorganic minerals; and extracting the various inorganic minerals from the micron CT scan image of each of the small core samples based on the micron CT scan image characteristics of the various inorganic minerals in the surface feature region of each of the small core samples, and determining the proportions of the various inorganic minerals in inorganic mineral, so that the determination of the inorganic mineral composition is achieved (the mineral types of the inorganic mineral and the proportions of the various inorganic minerals are determined);
I: for each of the small core samples, determining various inorganic minerals in the surface feature region based on the micron CT scan image and the XRF scan image of the surface feature region, and extracting the various minerals from the micron CT scan image based on the micron CT scan image characteristics of the various minerals in the surface feature region to determine inorganic mineral distribution (as shown in FIG. 3); and
J: obtaining a nuclear magnetic resonance image of each of the small core samples; based on the nuclear magnetic resonance image of each of the small core samples, observing water in each of the small core samples, and determining the distribution of water in each of the small core samples; and determining water-binding and oil-bearing characteristics of pores in each of the small core samples in combination with the micron CT scan image and the XRF scan image of each of the small core samples.

### Example 3

This example provides a method for core sample analysis. The method is configured to analyze the meter-sized core of a cap rock, and specifically comprises:
A: performing dual-energy spiral CT scanning with a standard sample on a meter-sized large core to be analyzed of a cap rock to obtain a dual-energy spiral CT scan image of each layer of the meter-sized large core to be analyzed; and determining the relative atomic number and average density of each layer of the meter-sized large core to be analyzed based on the dual-energy spiral CT scan image of the meter-sized large core to be analyzed, wherein the relative atomic number corresponds to the CT characterization value of a low-energy CT scan image in the dual-energy spiral CT scan image, and the average density corresponds to the CT characterization value of a high-energy CT scan image in the dual-energy spiral CT scan image; and
determining a typical rock section in the meter-sized large core to be analyzed based on the average density and relative atomic number of each layer of the meter-sized large core to be analyzed, and determining a plurality of small core samples (centimeter-sized or millimeter-sized) from the typical rock section;
B: obtaining the mass, volume and density of each of the small core samples;
C: performing feature region scanning on the surface of each of the small core samples through Micron XRF analysis to obtain an XRF scan image of the surface feature region of each of the small core samples; and performing dual-energy scanning on each of the small core samples by a micron X-ray microscope (Micron CT) to obtain a micron CT scan image of each of the small core samples;
D: determining the elemental composition of the surface feature region of each of the small core samples and the mass fraction of each element (including primary organic matter elements such as carbon W_{C}, sulfur W_{S}, nitrogen W_{N}, and oxygen W_{O}, and trace elements such as iron W_{Fe}, copper W_{Cu}, magnesium W_{Mg}, zinc W_{Zn}, molybdenum W_{Mu}, nickel W_{Ni}, and mercury W_{Hg}) based on the XRF scan image of the surface feature region of each of the small core samples;
E: determining organic matter, pores, and inorganic mineral in the surface feature region of each of the small core samples based on the micron CT scan image and the XRF scan image of the surface feature region of each of the small core samples; extracting the organic matter, the pores, and the inorganic mineral from the micron CT scan image of each of the small core samples based on the micron CT scan image characteristics of the organic matter, pores and inorganic mineral in the surface feature region of each of the small core samples to determine the organic matter distribution, porosity distribution and inorganic mineral distribution of each of the small core samples;
determining the volume of the organic matter, the volume of the pores, and the volume of the inorganic mineral in each of the small core samples based on results of the extraction of the organic matter, the pores, and the inorganic mineral from the micron CT scan image of each of the small core samples in combination with the volume of each of the small core samples, and determining the organic matter volume content, porosity, and inorganic mineral volume content of each of the small core samples;
determining the organic matter density (based on a scanned standard sample), mass and relative atomic number (based on a scanned standard sample) of each of the small core samples by using the average grayscale of a micron CT scan image of the organic matter of each of the small core samples, and determining the average density and average relative atomic number of the organic matter through weighted averaging to serve as the organic matter density and the organic matter relative atomic number of the meter-sized large core to be analyzed; and
determining the mass contents of carbon, oxygen, sulfur, and nitrogen in the organic matter of each of the small core samples based on the XRF scan image of the organic matter in the surface feature region of each of the small core samples, and determining the average mass content of carbon, average mass content of oxygen, average mass content of sulfur and average mass content of nitrogen in the organic matter through weighted averaging to serve as the mass contents of carbon, oxygen, sulfur, and nitrogen in the organic matter of the meter-sized large core to be analyzed;
F: determining a first CT grayscale value and a second CT grayscale value of each layer of the meter-sized large core to be analyzed based on the dual-energy spiral CT scan image of the meter-sized large core to be analyzed, wherein the first CT grayscale value corresponds to a CT grayscale value from the low-energy CT scan image in the dual-energy spiral CT scan image, and the second CT grayscale value corresponds to a CT grayscale value from the high-energy CT scan image in the dual-energy spiral CT scan image;
determining the contribution weight of the organic matter content per unit volume to the first CT grayscale value, the contribution weight of the pore content per unit volume to the first CT grayscale value, the contribution weight of the inorganic mineral content per unit volume to the first CT grayscale value, the contribution weight of the organic matter content per unit volume to the second CT grayscale value, the contribution weight of the pore content per unit volume to the second CT grayscale value, and the contribution weight of the inorganic mineral content per unit volume to the second CT grayscale value based on the organic matter volume content, porosity, and inorganic mineral volume content of each of the small core samples in combination with the first CT grayscale value and the second CT grayscale value of each layer of the meter-sized large core to be analyzed corresponding to each of the small core samples, wherein the contribution weight of the organic matter content per unit volume to the first CT grayscale value, the contribution weight of the pore content per unit volume to the first CT grayscale value, the contribution weight of the inorganic mineral content per unit volume to the first CT grayscale value, the contribution weight of the organic matter content per unit volume to the second CT grayscale value, the contribution weight of the pore content per unit volume to the second CT grayscale value, and the contribution weight of the inorganic mineral content per unit volume to the second CT grayscale value satisfy the following equations:

$$C {T}_{x} = {X}_{1} \cdot {V}_{1} + {X}_{2} \cdot {V}_{2} + {X}_{3} \cdot {V}_{3}$$

$${CT}_{Y} = {Y}_{1} \cdot {V}_{1} + {Y}_{2} \cdot {V}_{2} + {Y}_{3} \cdot {V}_{3}$$

where CTₓ is the first CT grayscale value; X₁ is the contribution weight of the organic matter content per unit volume to the first CT grayscale value; V₁ is the organic matter volume content; X₂ is the contribution weight of the pore content per unit volume to the first CT grayscale value; V₂ is the porosity; X₃ is the contribution weight of the inorganic mineral content per unit volume to the first CT grayscale value; V₃ is the inorganic mineral volume content; CT_{Y} is the second CT grayscale value; Y₁ is the contribution weight of the organic matter content per unit volume to the second CT grayscale value; Y₂ is the contribution weight of the pore content per unit volume to the second CT grayscale value; and Y₃ is the contribution weight of the inorganic mineral content per unit volume to the second CT grayscale value; and
determining the organic matter volume content, porosity, and inorganic mineral volume content of each layer of the meter-sized large core to be analyzed based on the first CT grayscale value and the second CT grayscale value of each layer of the meter-sized large core to be analyzed in combination with the contribution weight of the organic matter content per unit volume to the first CT grayscale value, the contribution weight of the pore content per unit volume to the first CT grayscale value, the contribution weight of the inorganic mineral content per unit volume to the first CT grayscale value, the contribution weight of the organic matter content per unit volume to the second CT grayscale value, the contribution weight of the pore content per unit volume to the second CT grayscale value, and the contribution weight of the inorganic mineral content per unit volume to the second CT grayscale value, and determining the organic matter volume content distribution, porosity distribution and inorganic mineral volume content distribution in the meter-sized large core to be analyzed;
F: determining various inorganic minerals in the surface feature region of each of the small core samples based on the micron CT scan image and the XRF scan image of the surface feature region of each of the small core samples; determining the elemental composition and the content of each element of the various inorganic minerals (including trace elements such as iron, copper, magnesium, zinc, molybdenum, nickel, and mercury) based on XRF scan images of the various inorganic minerals in the surface feature region of each of the small core samples; determining the mineral types of the various inorganic minerals based on the elemental composition and the content of each element of the various inorganic minerals; and extracting the various inorganic minerals from the micron CT scan image of each of the small core samples based on the micron CT scan image characteristics of the various inorganic minerals in the surface feature region of each of the small core samples, and determining the proportions of the various inorganic minerals in inorganic mineral, so that the determination of the inorganic mineral composition is achieved (the mineral types of the inorganic mineral and the proportions of the various inorganic minerals are determined);
G: for each of the small core samples, determining various inorganic minerals in the surface feature region based on the micron CT scan image and the XRF scan image of the surface feature region, and extracting the various minerals from the micron CT scan image based on the micron CT scan image characteristics of the various minerals in the surface feature region to determine inorganic mineral distribution;
H: determining the permeability of each of the small core samples; and
I: for each of the small core samples, performing displacement by using a high-concentration KI solution, drying the sample so that KI crystals will remain in the throats of the connected pores that cannot be observed by ordinary imaging, and then performing micron CT scanning to observe a seepage path.

The above descriptions are only preferred embodiments of the present invention and are not intended to limit the present invention. Any modifications, equivalent substitutions, improvements, or the like made within the spirit and principle of the present invention should be included within the protection scope of the present invention.

1. A method for core sample analysis, comprising steps of:
obtaining a dual-energy spiral computed tomography (CT) scan image of a meter-sized large core to be analyzed; and determining at least three small core samples in the meter-sized large core to be analyzed, wherein the dual-energy spiral CT scan image comprises a low-energy CT scan image and a high-energy CT scan image;
obtaining an XRF scan image of a surface feature region of each of the small core samples and a micron CT scan image of each of the small core samples;
determining the organic matter volume content, porosity, and inorganic mineral volume content of each of the small core samples by using the micron CT scan image of each of the small core samples and the XRF scan image of the surface feature region of each of the small core samples; and
determining the organic matter volume content distribution and/or porosity distribution and/or inorganic mineral volume content distribution in the meter-sized large core to be analyzed, based on the dual-energy spiral CT scan image of the meter-sized large core to be analyzed in combination with the organic matter volume content, porosity, and inorganic mineral volume content of each of the small core samples.

2. The method according to claim 1, wherein the step of determining the organic matter volume content, porosity, and inorganic mineral volume content of each of the small core samples by using the micron CT scan image of each of the small core samples and the XRF scan image of the surface feature region of each of the small core samples comprises:
determining the organic matter distribution, porosity distribution, and inorganic mineral distribution of each of the small core samples by using the micron CT scan image of each of the small core samples and the XRF scan image of the surface feature region of each of the small core samples; and
determining the organic matter volume content, porosity, and inorganic mineral volume content of each of the small core samples based on the organic matter distribution, porosity distribution, and inorganic mineral distribution of each of the small core samples.

3. The method according to claim 2, wherein the organic matter distribution, porosity distribution, and inorganic mineral distribution of each of the small core samples are determined by
determining organic matter, pores, and inorganic mineral in the surface feature region of a small core sample based on the micron CT scan image of the surface feature region of the small core sample and the XRF scan image of the surface feature region of the small core sample; and
extracting the organic matter, the pores, and the inorganic mineral from the micron CT scan image of the small core sample based on the micron CT scan image characteristics of the organic matter, pores, and inorganic mineral in the surface feature region of the small core sample, and determining the organic matter distribution, porosity distribution, and inorganic mineral distribution of the small core sample.

4. The method according to claim 1, wherein the step of determining the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples comprises:
determining a first CT characterization value and a second CT characterization value of each layer of the meter-sized large core to be analyzed based on the dual-energy spiral CT scan image of the meter-sized large core to be analyzed, wherein the first CT characterization value corresponds to a CT characterization value from the low-energy CT scan image in the dual-energy spiral CT scan image, and the second CT characterization value corresponds to a CT characterization value from the high-energy CT scan image in the dual-energy spiral CT scan image;
determining the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value, based on the organic matter volume content, porosity, and inorganic mineral volume content of each of the small core samples in combination with the first CT characterization value and the second CT characterization value of each layer of the meter-sized large core to be analyzed corresponding to each of the small core samples; and
determining the organic matter volume content and/or porosity and/or inorganic mineral volume content of each layer of the meter-sized large core to be analyzed, based on the first CT characterization value and the second CT characterization value of each layer of the meter-sized large core to be analyzed in combination with the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value, and determining the organic matter volume content distribution and/or porosity distribution and/or inorganic mineral volume content distribution in the meter-sized large core to be analyzed.

5. The method according to claim 4, wherein the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value satisfy the following equations:
$$C {T}_{x} = {X}_{1} \cdot {V}_{1} + {X}_{2} \cdot {V}_{2} + {X}_{3} \cdot {V}_{3}$$
$${CT}_{Y} = {Y}_{1} \cdot {V}_{1} + {Y}_{2} \cdot {V}_{2} + {Y}_{3} \cdot {V}_{3}$$
wherein CTₓ is the first CT characterization value; X₁ is the contribution weight of the organic matter content per unit volume to the first CT characterization value; V₁ is the organic matter volume content; X₂ is the contribution weight of the pore content per unit volume to the first CT characterization value; V₂ is the porosity; X₃ is the contribution weight of the inorganic mineral content per unit volume to the first CT characterization value; V₃ is the inorganic mineral volume content; CT_{Y} is the second CT characterization value; Y₁ is the contribution weight of the organic matter content per unit volume to the second CT characterization value; Y₂ is the contribution weight of the pore content per unit volume to the second CT characterization value; and Y₃ is the contribution weight of the inorganic mineral content per unit volume to the second CT characterization value.

6. The method according to claim 1, further comprising: determining the total organic carbon of each of the small core samples by using the micron CT scan image of each of the small core samples and the XRF scan image of the surface feature region of each of the small core samples, wherein the total organic carbon of each of the small core samples is determined by:
determining the organic matter in the surface feature region of the small core sample based on the micron CT scan image of the surface feature region of the small core sample and the XRF scan image of the surface feature region of the small core sample;
extracting organic matter from the micron CT scan image of the small core sample based on the micron CT scan image characteristics of the organic matter in the surface feature region of the small core sample, and determining the volume of the organic matter in the small core sample further based on the volume of the small core sample;
determining the density of the organic matter by using the micron CT scan image of the organic matter in the small core sample;
determining the carbon element mass content of the organic matter based on the XRF scan image of the organic matter in the surface feature region of the small core sample; and
determining the total organic carbon (TOC) of the small core sample by using the volume, density and carbon element mass content of the organic matter in combination with the mass of the small core sample,
wherein the TOC is determined by the following equations:
$$TOC = {m}_{C} \div {m}_{2}$$
$${m}_{O} = {p}_{0} \times {V}_{O}$$
$${m}_{C} = {m}_{O} \times {V}_{OC}$$
wherein TOC is the total organic carbon; m_{C} is the total mass of organic carbon; m_{O} is the total mass of the organic matter; m₂ is the mass of a core sample to be analyzed; ρ_{O} is the density of the organic matter; V_{O} is the volume of the organic matter; and W_{OC} is the carbon element mass content of the organic matter.

7. The method according to claim 1, further comprising a step of determining the organic matter carbon content distribution in the meter-sized large core to be analyzed by using the micron CT scan image of each of the small core samples and the XRF scan image of the surface feature region of each of the small core samples in combination with the dual-energy spiral CT scan image of the meter-sized large core to be analyzed and the organic matter volume content distribution in the meter-sized large core to be analyzed, wherein the step of determining the organic matter carbon content distribution in the meter-sized large core comprises:
determining the organic matter in the surface feature region of each of the small core samples based on the micron CT scan image and the XRF scan image of the surface feature region of each of the small core samples;
determining the density of the organic matter by using the micron CT scan image of the organic matter in each of the small core samples;
determining the carbon element mass content of the organic matter based on the XRF scan image of the organic matter in the surface feature region of each of the small core samples;
determining the average density of each layer of the meter-sized large core to be analyzed by using the dual-energy spiral CT scan image of the meter-sized large core to be analyzed; and
determining the total organic carbon of each layer of the meter-sized large core to be analyzed by using the density and carbon element mass content of the organic matter in combination with the average density and the organic matter volume content of each layer of the meter-sized large core to be analyzed,
wherein the total organic carbon of each layer of the meter-sized large core to be analyzed is determined by the following equation:
$${TOC}_{i} = {ρ}_{0} \cdot {V}_{1 i} \cdot {W}_{OC} \div {ρ}_{i}$$
wherein TOCᵢ is the total organic carbon of the i^{th} layer of the meter-sized large core to be analyzed; ρ_{O} is the density of the organic matter; V₁ᵢ is the organic matter volume content of the i^{th} layer of the meter-sized large core to be analyzed; W_{OC} is the carbon element mass content of the organic matter; and ρᵢ is the average density of the i^{th} layer of the meter-sized large core to be analyzed.

8. The method according to claim 1, further comprising a step of determining the type of the organic matter by using the micron CT scan image of each of the small core sample and the XRF scan image of the surface feature region of each of the small core samples.

9. The method according to claim 8, wherein the type of the organic matter is determined by:
determining the organic matter in the surface feature region of each of the small core samples based on the micron CT scan image and the XRF scan image of the surface feature region of each of the small core samples;
determining the carbon element mass content and oxygen element mass content of the organic matter based on the XRF scan image of the organic matter in the surface feature region of each of the small core samples,
determining the number ratio of oxygen atoms to carbon atoms in the organic matter based on the carbon element mass content and oxygen element mass content of the organic matter; and
determining the type of the organic matter by using the number ratio of oxygen atoms to carbon atoms in the organic matter,
wherein the number ratio of oxygen atoms to carbon atoms when the type of the organic matter is Type I kerogen<the number ratio of oxygen atoms to carbon atoms when the type of the organic matter is Type II kerogen<the number ratio of oxygen atoms to carbon atoms when the type of the organic matter is Type III kerogen.

10. The method according to claim 8, wherein the type of the organic matter is determined by:
determining the organic matter in the surface feature region of each of the small core samples based on the micron CT scan image and the XRF scan image of the surface feature region of each of the small core samples;
determining the carbon element mass content and hydrogen element mass content of the organic matter based on the XRF scan image of the organic matter in the surface feature region of each of the small core samples;
determining the number ratio of hydrogen atoms to carbon atoms in the organic matter based on the carbon element mass content and hydrogen element mass content of the organic matter; and
determining the type of the organic matter by using the number ratio of hydrogen atoms to carbon atoms in the organic matter,
wherein the number ratio of hydrogen atoms to carbon atoms when the type of the organic matter is Type I kerogen or Type II kerogen>the number ratio of hydrogen atoms to carbon atoms when the type of the organic matter is Type III kerogen.

11. The method according to claim 1, further comprising a step of determining the organic matter maturity by using the micron CT scan image of each of the small core samples and the XRF scan image of the surface feature region of each of the small core samples, wherein the organic matter maturity is determined by:
determining the organic matter in the surface feature region of each of the small core samples based on the micron CT scan image and the XRF scan image of the surface feature region of each of the small core samples;
determining the elemental composition and the content of each element of the organic matter based on the XRF scan image of the organic matter in the surface feature region of each of the small core samples,
determining the average atomic number of the organic matter based on the elemental composition and the content of each element of the organic matter; and
determining the vitrinite reflectance of the organic matter based on the average atomic number of the organic matter.

12. The method according to claim 11, wherein the determining the vitrinite reflectance of the organic matter based on the average atomic number of the organic matter comprises:
obtaining a relational expression between the vitrinite reflectance and the average atomic number of the organic matter; and
determining the vitrinite reflectance of the organic matter by using the average atomic number of the organic matter and the relational expression between the vitrinite reflectance and the average atomic number of the organic matter.

13. The method according to claim 1, further comprising a step of determining the inorganic mineral composition by using the micron CT scan image and the XRF scan image of each of the small core samples of the surface feature region of each of the small core samples, wherein the inorganic mineral composition is determined by:
determining various inorganic minerals in the surface feature region of each of the small core samples based on the micron CT scan image and the XRF scan image of the surface feature region of each of the small core samples;
determining the elemental composition and the content of each element of the various inorganic minerals based on XRF scan images of the various inorganic minerals in the surface feature region of each of the small core samples; and determining the mineral types of the various inorganic minerals based on the elemental composition and the content of each element of the various inorganic minerals; and
extracting the various inorganic minerals from the micron CT scan image of each of the small core samples based on the micron CT scan image characteristics of the various inorganic minerals in the surface feature region of each of the small core samples, and determining the proportions of the various inorganic minerals in inorganic mineral.

14. A system for core sample analysis, comprising:
a large-core data acquisition module, configured to obtain a dual-energy spiral CT scan image of a meter-sized large core to be analyzed; and to determine at least three small core samples in the meter-sized large core to be analyzed, wherein the dual-energy spiral CT scan image comprises a low-energy CT scan image and a high-energy CT scan image;
a small-core data acquisition module, configured to obtain an XRF scan image of a surface feature region of each of the small core samples and a micron CT scan image of each of the small core samples;
a small-core parameter determination module, configured to determine the organic matter volume content, porosity, and inorganic mineral volume content of each of the small core samples by using the micron CT scan image of each of the small core samples and the XRF scan image of the surface feature region of each of the small core samples; and
a large-core parameter determination module, configured to determine the organic matter volume content distribution and/or porosity distribution and/or inorganic mineral volume content distribution in the meter-sized large core to be analyzed based on the dual-energy spiral CT scan image of the meter-sized large core to be analyzed in combination with the organic matter volume content, porosity, and inorganic mineral volume content of each of the small core samples.

15. The system according to claim 14, wherein the large-core parameter determination module comprises:
a CT characterization value determination submodule, configured to determine a first CT characterization value and a second CT characterization value of each layer of the meter-sized large core to be analyzed based on the dual-energy spiral CT scan image of the meter-sized large core to be analyzed, wherein the first CT characterization value corresponds to a CT characterization value from the low-energy CT scan image in the dual-energy spiral CT scan image, and the second CT characterization value corresponds to a CT characterization value from the high-energy CT scan image in the dual-energy spiral CT scan image;
a contribution weight determination submodule, configured to determine the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value, based on the organic matter volume content, porosity, and inorganic mineral volume content of each of the small core samples in combination with the first CT characterization value and the second CT characterization value of each layer of the meter-sized large core to be analyzed corresponding to each of the small core samples; and
a large-core parameter determination submodule, configured to determine the organic matter volume content and/or porosity and/or inorganic mineral volume content of each layer of the meter-sized large core to be analyzed, based on the first CT characterization value and the second CT characterization value of each layer of the meter-sized large core to be analyzed in combination with the contribution weight of the organic matter content per unit volume to the first CT characterization value, the contribution weight of the pore content per unit volume to the first CT characterization value, the contribution weight of the inorganic mineral content per unit volume to the first CT characterization value, the contribution weight of the organic matter content per unit volume to the second CT characterization value, the contribution weight of the pore content per unit volume to the second CT characterization value, and the contribution weight of the inorganic mineral content per unit volume to the second CT characterization value, and determining the organic matter volume content distribution and/or porosity distribution and/or inorganic mineral volume content distribution in the meter-sized large core to be analyzed.

16. The system according to claim 14, further comprising:
a small-core TOC determination module, configured to determine the total organic carbon of each of the small core samples by using the micron CT scan image of each of the small core samples and the XRF scan image of the surface feature region of each of the small core samples, wherein the small-core TOC determination module is specifically configured to determine the total organic carbon of each of the small core samples by:
determining the organic matter in the surface feature region of a small core sample based on the micron CT scan image of the surface feature region of the small core sample and the XRF scan image of the surface feature region of the small core sample;
extracting organic matter from the micron CT scan image of the small core sample based on the micron CT scan image characteristics of the organic matter in the surface feature region of the small core sample, and determining the volume of the organic matter in the small core sample further based on the volume of the small core sample;
determining the density of the organic matter by using the micron CT scan image of the organic matter in the small core sample;
determining the carbon element mass content of the organic matter based on the XRF scan image of the organic matter in the surface feature region of the small core sample; and
determining the total organic carbon (TOC) of the small core sample by using the volume, density, and carbon element mass content of the organic matter in combination with the mass of the small core sample,
wherein the TOC is determined by the following equations:
$$TOC = {m}_{C} \div {m}_{2}$$
$${m}_{O} = {p}_{0} \times {V}_{O}$$
$${m}_{C} = {m}_{O} \times {V}_{OC}$$
wherein TOC is the total organic carbon; m_{C} is the total mass of organic carbon; m_{O} is the total mass of the organic matter; m₂ is the mass of a core sample to be analyzed; ρ_{O} is the density of the organic matter; V_{O} is the volume of the organic matter; and W_{OC} is the carbon element mass content of the organic matter.

17. The system according to claim 14, further comprising:
a TOC distribution determination module, configured to determine the organic matter carbon content distribution in the meter-sized large core to be analyzed by using the micron CT scan image of each of the small core samples and the XRF scan image of the surface feature region of each of the small core samples in combination with the dual-energy spiral CT scan image of the meter-sized large core to be analyzed and the organic matter volume content distribution in the meter-sized large core to be analyzed,
wherein the TOC distribution determination module is configured to determine the organic matter carbon content distribution in the meter-sized large core to be analyzed by:
determining the organic matter in the surface feature region of each of the small core samples based on the micron CT scan image and the XRF scan image of the surface feature region of each of the small core samples;
determining the density of the organic matter by using the micron CT scan image of the organic matter in each of the small core samples;
determining the carbon element mass content of the organic matter based on the XRF scan image of the organic matter in the surface feature region of each of the small core samples;
determining the average density of each layer of the meter-sized large core to be analyzed by using the dual-energy spiral CT scan image of the meter-sized large core to be analyzed; and
determining the total organic carbon of each layer of the meter-sized large core to be analyzed by using the density and carbon element mass content of the organic matter in combination with the average density and the organic matter volume content of each layer of the meter-sized large core to be analyzed,
wherein the total organic carbon of each layer of the meter-sized large core to be analyzed is determined by the following equation:
$${TOC}_{i} = {ρ}_{0} \cdot {V}_{1 i} \cdot {W}_{OC} \div {ρ}_{i}$$
wherein TOCi is the total organic carbon of the i^{th} layer of the meter-sized large core to be analyzed; ρ_{O} is the density of the organic matter; V₁ᵢ is the organic matter volume content of the i^{th} layer of the meter-sized large core to be analyzed; W_{OC} is the carbon element mass content of the organic matter; and ρᵢ is the average density of the i^{th} layer of the meter-sized large core to be analyzed.

18. (canceled)

19. A computer-readable storage medium; having a computer program stored therein, wherein when the computer program is executed by a processor, the steps of the method for core sample analysis according to claim 1 are implemented.
